(19) United States
(12) Patent Application Publication
(10) Pub. No.: US 2009/0269393 A1
(43) Pub. Date: Oct. 29, 2009

(54) Chewable Bilayer Tablet Formulation

(75) Inventors: Namdev Kashid, Maharashtra (IN); Pradeep Chouhan, Madhya Pradesh (IN); Gour Mukherji, Haryana (IN)

Correspondence Address: COHEN, PONTANI, LIEBERMAN & PAVANE LLP, 551 FIFTH AVENUE, SUITE 1210, NEW YORK, NY, 10176, US

(73) Assignee: JUBLIANT ORGANOSYS LIMITED, Noida (IN)

(21) Appl. No.: 12/304,511

(22) PCT Filed: Jun. 12, 2007
(86) PCT No.: PCT/IN2007/000234

§ 371 Date: Dec. 12, 2008

(30) Foreign Application Priority Data
Jun. 12, 2006 (IN) 1399/DEL/2006

(51) Int. Cl. A61K 9/20; A61P 37/08

(52) U.S. Cl. 424/441

(57) ABSTRACT

Disclosed herein is a tablet formulation of an objectionable tasting drug cetirizine or its pharmaceutically acceptable salt in a form of chewable bilayer tablet, wherein the formulation comprises said cetirizine, a combination of water-insoluble and water-soluble polymer in a ratio of about 1:0.5 to about 1:5 and a low molecular weight polyol, wherein the molar ratio of the low molecular weight polyol to cetirizine is more than 10, and the inactive formulation layer comprises beta-cyclodextrin and other pharmaceutically acceptable excipients. Further, the present invention provides a process for preparing the formulation.

### PRIORITY CLAIM

This is a U.S. national stage of application No. PCT/IN2007/000234, filed on Jun. 12, 2007. Priority is claimed on the following application(s): Country: India, Application No.: 1399/DEL/2006, Filed: Jun. 12, 2006, the content of which is/are incorporated here by reference.

### FIELD OF THE INVENTION

In general, the present invention relates to a pharmaceutical formulation in the form of a chewable bilayer tablet of objectionable tasting drugs. More particularly, the present invention provides a pharmaceutical formulation in the form of a palatable chewable bilayer tablet comprising cetirizine or its pharmaceutically acceptable salt and a process for preparation of said formulation.

### BACKGROUND OF THE INVENTION

Cetirizine, which is chemically is known as (+)-[2-[4-[(4-chlorophenyl)phenylmethyl]-1-piperazinyl]ethoxy]acetic acid dihydrochloride, also known as cetirizine dihydrochloride, has been approved by the USFDA for use in seasonal allergic rhinitis, perennial allergic rhinitis and chronic urticaria. The drug is orally active and has excellent antiallergic and antihistaminic properties.

Chewable tablets are widely used in the pharmaceutical industry for patients, such as children, who have difficulty swallowing conventional tablets or capsules. Hence, chewable tablets are often utilized to improve patient compliance in pediatric and sometimes in geriatric patients also. However, patient compliance may be limited in the situation where the drug to be administered is bitter, bad tasting or in some manner unpleasant especially to children.

Palatability and “mouth feel” are extremely important factors in formulating chewable tablets of bitter tasting drugs like cetirizine or its pharmaceutically acceptable salts. Cetirizine hydrochloride is known to have a formulation disadvantage in that it has an inherently unpleasant mouth feel and unpalatable bitter taste. Accordingly, the practical value of cetirizine formulations is substantially diminished since patients finding them objectionable may fail to take them as prescribed. Therefore, it is a challenging task for the formulation scientist to provide a highly palatable chewable tablet of cetirizine without compromising the bioavailabilty and stability of the drug.

Many approaches have been attempted to mask the taste of bitter drugs which are administered orally as value added products, like chewable tablets, orally disintegrating tablets, suspensions and syrups. Below are some of the approaches for taste-masking, particularly in chewable tablets.

U.S. Pat. No. 3,558,600 describes a method for masking the bitter taste of antibistaminic agents belonging to the substituted 1-(p-chloro-benzhydryl)piperazine family, which consists of converting the active substance from its salt form to the free base form, with a long-chain alkyl sulfate, for example, such as stearyl sulfate. This method has its own limitations like poor solubility and reduced absorption of drug. Besides, this approach cannot be successful for highly bitter drugs.

Damani et al., in EPO Patent No. 212641 discloses a chewable medicinal tablet wherein the active ingredient (which can be dimenhydrinate) is embedded in a matrix of copolymers of methylmethacrylate acid or esters for taste masking.

U.S. Pat. No. 5,869,095 describes a chewable tablet or lozenge with an effervescent action comprising acid and carbonate particles of an effervescent base that are coated with a soluble hydrocolloid. Such tablets generate an objectionable fizzy feeling in the mouth.

Another approach as described in U.S. Pat. No. 5,084,278 is coating the drug with polymers to mask their unpleasant taste. However, the forces used to compress these tablets can fracture the polymer coating, which reduces the effectiveness of the taste-masking system. Moreover, the crushing of the tablet by the teeth of the patient can also expose the drug in the patient's mouth, leading to bitter mouth-feel and poor patient compliance.

U.S. Pat. No. 6,471,991 assigned to McNeil PPC relates to soft, convex-shaped compressed chewable tablets and a process for preparing such tablets. It is mentioned that convex-shaped, chewable tablets are softer than conventional chewable tablets, which results in improvements in product taste, mouth feel and ease of chewing. These compositions require very low compression forces, resulting in tablets having lower hardness leading to problems associated with conventional bulk handling equipment and packaging.

A survey of the prior art reveals that whenever there is effective taste masking of an objectionable tasting drug in the dosage form, it usually compromises the dissolution rate and bioavailability of the drug from the dosage form or vice versa.

U.S. Patent Application 2005/0038039 for cetirizine bilayer chewable tablets teaches that when polyols of low molecular weight (molecular weight less than 950) are used with cetirizine in the molar ratio of polyol to cetirizine above 10, it leads to undesired reaction product. Hence, the polyol and the drug have been taken in separate layers of the bilayer tablet.

The present invention provides the solution to this long-existing problem by providing a stable taste-masked chewable composition of cetirizine or its pharmaceutically acceptable salts in the form of the bilayer tablet.

### SUMMARY OF THE INVENTION

It is a principal aspect of the present invention to provide a palatable chewable bilayer tablet comprises cetirizine or its pharmaceutically acceptable salts, wherein said palatability and mouth feel effect of the tablet is achieved along with desired bioavailability and stability of said drug.

In accordance with another aspect of the present invention, there is provided a stable pharmaceutical formulation in the form of a palatable chewable bilayer tablet, wherein said formulation comprising a first active formulation layer having an effective ratio of water-soluble polymer and water-insoluble polymer, low molecular weight polyols (molecular weight less than 950) and other optional pharmaceutically acceptable excipients, wherein the combination of said excipients provides better taste-masking along with desired release of the drug and the second inactive formulation layer has beta-cyclodextrin and other pharmaceutically acceptable excipients, wherein beta-cyclodextrin is not in intimate contact with cetirizine or its pharmaceutically acceptable salts of the active formulation layer.

In accordance with one other aspect of the present invention, there is provided a stable pharmaceutical formulation in the form of a palatable chewable bilayer tablet, wherein said formulation comprises a first active formulation having water-soluble polymer and water-insoluble polymer in a range of ratios from 1:0.5 to 1:5, preferably 1:1.5 to 1:3, low molecular weight polyols (molecular weight less than 950) and other optional pharmaceutically acceptable excipients, wherein the ratio of low molecular weight polyol to drug is more than 10.

In accordance with yet another aspect of the present invention, there is provided a stable pharmaceutical formulation in the form of a palatable chewable bilayer tablet, wherein the low molecular weight polyols (molecular weight less than 950) and said drug cetirizine are in a single layer as a first active formulation layer having a molar ratio of polyol to drug of more than 10, wherein the formulation provides an effective taste-masking along with desired release of said drug without formation of undesired reaction products.

In accordance with a further aspect of the present invention, there is provided a process for manufacturing a pharmaceutical composition in the form of a palatable oral chewable bilayer tablet comprising the steps of (a) preparing the active formulation layer comprising cetirizine, filler granules and other pharmaceutically acceptable excipients, (b) preparing an inactive formulation comprising beta-cyclodextrin and (c) combining the active formulation and inactive formulation using a bilayer tablet machine to get the bilayered tablet such that said cyclodextrin is not in intimate contact with cetirizine or its pharmaceutically acceptable salts.

### DETAILED DESCRIPTION OF THE INVENTION

While this specification concludes with claims particularly pointing out and distinctly claiming that which is regarded as the invention, it is anticipated that the invention can be more readily understood through reading the following detailed description of the invention and studying of the included examples.

The present invention provides a pharmaceutical formulation in the form of a palatable chewable bilayer tablet of highly objectionable tasting drug cetirizine or its pharmaceutically acceptable salts and a process for manufacturing the same.

The term “chewable tablet” as used herein refers to a solid dosage form, which can be taken by mouth and crushed into smaller pieces before swallowing.

The term “therapeutically effective amount” as herein used is the amount or quantity of an active ingredient which is sufficient to elicit the required or desired therapeutic response.

The term “pharmaceutically acceptable excipient” as used herein is intended to denote any material which is inert in the sense that it substantially does not have any therapeutic and/or prophylactic effect per se. Such an excipient may be added with the purpose of making it possible to obtain a pharmaceutical composition which has acceptable technical properties.

The term “optional” or “optionally” means that subsequently described excipient or circumstances may or may not be present, so that the description includes instances where the excipient or circumstances are present or instances where they are not. The term “granule” in this specification is a granulated material in which powdered material is grown to be with a fixed particle diameter using a binder according to a known granulation method.

The term Cₘₐₓ as used herein means the maximum plasma/blood cetirizine concentration achieved after oral administration of the cetirizine chewable tablets.

The term AUC (area under curve) as used herein indicates the total amount of cetirizine absorbed by the bloodstream in a predetermined time, generally 24 hours. AUC is a measure of bioavailability, which is calculated by integrating plasma concentration levels of cetirizine with respect to time.

Specifically, the present invention provides a stable pharmaceutical composition in the form of a palatable chewable bilayer tablet comprising, (a) a first distinct layer made with active formulation, which comprises a therapeutically effective amount of cetirizine or its pharmaceutically acceptable salts, a combination of water-soluble polymer and water-insoluble polymer, low molecular weight polyols (molecular weight less than 950) and other optional pharmaceutically acceptable excipients, wherein the first active distinct layer provides the desired therapeutic response of the cetirizine or its pharmaceutically acceptable salts and wherein the ratio of water-soluble polymer to water-insoluble polymer ranges from 1:0.5 to 1:5, preferably 1:1.5 to 1:3 and wherein the ratio of low molecular weight polyol to drug is more than 10, and (b) a second distinct layer made with inactive formulation comprising beta-cyclodextrin and other pharmaceutically acceptable excipients, wherein beta-cyclodextrin is not in intimate contact with the cetirizine or its pharmaceutically acceptable salts of active formulation layer.

### Active Formulation

Active formulation refers to the distinct formulation containing the therapeutically effective amount of cetirizine or its pharmaceutically acceptable salts. Cetirizine or its pharmaceutically acceptable salts are used in an amount of about 0.01% to about 10% by weight based on the weight of the active formulation.

As used herein the term “drug” means cetrizine or its pharmaceutically acceptable salts.

The water-insoluble polymer of the active formulation includes, but is not limited to, ethyl cellulose, polyvinyl acetate, neutral copolymer based on ethylacrylate and methylmetacrylate (available under brand name Eudragit NE 30 D), copolymer of acrylic acid and methacrylic acid esters with quaternary ammonium compounds (available under the brand name Eudragit RS and RL), polyvinyl acetate phthalate, hydroxypropyl methylcellulose phthalate, hydroxypropyl methylcellulose succinate and shellac or combination thereof. The preferred water-insoluble polymer of the active formulation is ethyl cellulose.

The water-soluble polymer of the active formulation layer includes, but is not limited to, hydroxypropyl methylcellulose, hydroxypropyl cellulose, hydroxyethyl cellulose, polyvinyl alcohol, polyvinylpyrrolidone, methylcellulose, carboxymethyl cellulose and polyethylene glycols or combination thereof. The preferred water-soluble polymer of the active formulation is hydroxypropyl methylcellulose. The ratio of water-soluble polymer to water-insoluble polymer in the active formulation ranges from 1:0.5 to 1:5, and preferably 1:1.5 to 1:3. The ratio of water-soluble to water-insoluble polymer is dependent upon the degree to which the taste is to be masked. It is ascertained by skilled artisans that the ratio can be optimized by studying the taste and in-vitro release profiles of the active ingredient.

The water-insoluble polymer is used in an amount of about 5% to about 45% by weight based on the weight of the active formulation. The water-soluble polymer is used in an amount of about 3% to about 15% by weight based on the weight of the active formulation.

The term “water-soluble polymer” as used herein includes polymers which are freely permeable to water, whilst the term “water-insoluble polymer” as used herein includes polymers which are slightly permeable to water, as hereinafter indicated.

Patent application U.S. 2005/0038039 teaches that when polyols of low molecular weight (molecular weight less than 950) are processed along with the drug in the molar ratio of polyol to cetirizine above 10, this leads to an undesired reaction product due to the highly reactive nature of such polyols. In the composition of the present invention, low molecular weight polyol has been blended along with drug and said ratio has been more than 10, yet said composition is free of any undesired reaction products.

The term “low molecular weight polyol” as used herein refers to polyols having molecular weight less than 950. These polyols include mannitol, xylitol, sorbitol, dextrose, lactose and sucrose or combinations thereof. The preferred low molecular weight polyol in the present invention is mannitol.

Other optional pharmaceutically acceptable excipients of the active formulation layer may comprise fillers, neutralizing agents, binders, sweetening agents, plasticizers, glidants, lubricants, disintegrating agents, flavoring agents and coloring agents.

The non-limiting examples of fillers of the active formulation layer include microcrystalline cellulose, dicalcium phosphate, tricalcium phosphate, lactose, mannitol, xylitol, sorbitol, dextrose and sucrose or combination thereof. The preferred fillers are microcrystalline cellulose and mannitol. Microcrystalline cellulose is available under the brand name Avicel®. Mannitol is used as filler in the manufacture of chewable taste-masked dosage forms because of its negative heat of solution and sweetness. For the present invention, preferably granulated (referred to as “mannitol granules”) or spray dried mannitol (commercially available under the brand name Pearlitol SD200 from Roquette, France) is useful to achieve the desired mouth feel. Mannitol can be used in varied particle sizes without affecting the mouth feel. Higher average particle size mannitol suitable for direct compression can also be used. The filler can be used in an amount of about 10% to about 70% by weight based on the weight of the active formulation.

Neutralizing agents of the active formulation layer are selected from the group comprising sodium carbonate, sodium bicarbonate, calcium carbonate, magnesium carbonate and magnesium hydroxide. For the composition of the present invention, the preferred neutralizing agent is sodium carbonate. The neutralizing agent can be used in an amount of about 0 to about 2% by weight based on the weight of the active formulation.

Binders in the active formulation layer are selected from the group comprising polyvinylpyrrolidone (Kollidon K-30), hydroxypropyl methylcellulose, hydroxypropylcellulose, hydroxyethylcellulose, methylcellulose, liquid glucose, sucrose, maltodextrins, pregelatinized starch, sodium alginate, starch, tragacanth, zein, acacia, alginic acid, carbomer, carboxymethyl cellulose sodium, dextrin, ethylcellulose, magnesium aluminium silicate, gelatin and guar gum or combinations thereof. For the composition of the present invention, the preferred binder is polyvinylpyrrolidone (Kollidon K-30). The binder is used in an amount of about 1% to about 20% by weight based on the weight of the active formulation.

Disintegrating agents in the active formulation layer are selected from the group comprised of sodium starch glycolate, crosslinked polyvinyl pyrrolidone, croscarmellose sodium and low-substituted hydroxypropyl cellulose or combinations thereof. Preferred disintegrating agents are croscarmellose sodium and cross-linked polyvinylpyrrolidone (Polyplasdone-XL). The disintegrant is used in an amount of about 1% to about 20% by weight based on the weight of the active formulation.

Sweetening agents employed in the active formulation layer are selected from the group comprised of aspartame, acesulfame potassium, saccharin sodium, cyclamates, sucralose and other commercial artificial sweeteners well known to those of skilled in the art. For the composition of the present invention, the preferred sweetening agents are aspartame, acesulfame potassium or combinations thereof. Typically, the sweetener is used in an amount of less than 10% by weight based on the weight of the active formulation.

It is within the scope of the present invention to optionally include edible organic acids in the active formulation layer. Various examples of organic acids are known to persons skilled in the pharmaceutical art. The preferred organic acid is fumaric acid. Glidants added to the active formulation layer are selected from the group comprising talc, colloidal silicon dioxide and cornstarch or combinations thereof and other glidants well known to those of skilled in the art. The preferred glidant is colloidal silicon dioxide available under the brand name Aerosil-200. The glidant can be used in an amount of about 0.5% to about 2% by weight based on the weight of the active formulation.

Lubricants in the active formulation layer are selected from the group comprised of stearic acid, magnesium stearate, sodium stearylfumarate, sucrose ester of fatty acids, glyceryl behenate, polyethyleneglycol, glyceryl monostearate, glyceryl palmitostearate, hydrogenated castor oil and hydrogenated vegetable oil or combinations thereof. The preferred lubricant is magnesium stearate and can be used in an amount of about 0.5% to about 5% by weight based on the weight of the active formulation.

Flavoring agents are selected from the group comprising strawberry flavor, grape fruit flavor, orange flavor, vanilla cream flavor, raspberry flavor, banana flavor, watermelon flavor and peppermint oil or combinations thereof. Other artificial flavors known to those skilled in the art are also within the scope of this invention. The flavors can be used in an amount of about 0.1% to about 5% by weight based on the weight of the active formulation. Those skilled in the art will appreciate that the exact amounts will vary depending upon the strength of the particular flavoring agent used and will know bow to adjust the concentration to achieve the appropriate level of taste.

### Inactive Formulation

The inactive formulation layer of the present invention may include fillers, organic acids and other pharmaceutically acceptable excipients.

The non-limiting examples of fillers include mannitol, xylitol, lactose, sorbitol, dextrose, maltitol, sucrose, maltol, maltodextrin, modified starch and beta-cyclodextrin. Fillers can be used in an amount of about 75% to about 90% by weight based on the weight of the inactive formulation. For the composition of the present invention, the preferred fillers are mannitol and beta-cyclodextrin or combinations thereof. For the present invention, preferably, spray-dried mannitol (commercially available under the brand name Pearlitol SD200 from Roquette, France) or granulated mannitol (referred as “mannitol granules”) or directly compressible mannitol is useful to achieve the desired mouth feel. Preferably, for direct compression purposes, directly compressible beta-cyclodextrin can be used. Cyclodextrins are cyclic multicyclopyranose units connected by alpha-(1,4) linkages. The most widely known cyclodextrins are alpha, beta and gamma-cyclodextrins. The beta-cyclodextrin contained in the inactive formulation layer is not in intimate contact with cetirizine or its pharmaceutically acceptable salts present in the active formulation layer.

It is within the scope of the invention to use edible organic acids in the inactive formulation to provide the residual taste-masking effect. The organic acids suitable for use in the inactive layer can be selected from the group comprised of fumaric acid, citric acid, malic acid, succinic acid, ascorbic acid, maleic acid, tartaric acid and the like. Edible organic acid can be used in an amount of about 3% to about 10% by weight based on the weight of the inactive formulation.

Additionally, standard pharmaceutically acceptable excipients such as binders, disintegrants, flavors, sweeteners, lubricants and the like can be used in the inactive formulation to provide adequate compression and palatability. These excipients have already been elaborated in connection with the above-mentioned active formulation layer. Typical excipients include, for instance, binder (about 0% to about 5%), disintegrants (with maximum about 5%), flavors (about 0.1% to about 2%), sweeteners (about 0.5% to about 5%) and glidants (about 0.5% to about 2%) and said percentages are taken by weight of the inactive formulation. Those skilled in the art will appreciate that the amount of excipients may vary depending on the strength of particular excipients used and the level approved by regulatory authorities for use in pharmaceutical products.

The term “about” when used as a modifier for, or in conjunction with, a variable, is intended to convey that the numbers and ranges disclosed herein are flexible and that practice of the present invention by those skilled in the art using concentrations, amounts, contents and properties that are outside of the range or different from a single value, will achieve the desired result, namely palatable chewable bilayer formulation and methods for preparing and using such formulations.

A typical process for manufacturing the pharmaceutical composition of the present invention in the form of a palatable oral chewable bilayer tablet comprises the steps of (1) preparing the active formulation comprising cetirizine, filler granules and other pharmaceutically acceptable excipients, (2) preparing the inactive formulation comprising beta-cyclodextrin, and (3) combining the active formulation and inactive formulation using a bilayer tablet machine to get the bilayered tablet.

The formulations are obtained using various methods, such as wet granulation, direct compression, dry granulation, moulding, coating and other known methods. All these methods are well known in the art, and are described in detail, for example, by Lachman, et al., “The Theory and Practice of Industrial Pharmacy” which is incorporated by reference herein.

In one of the embodiments of the invention, the active substance is present in multiparticulate form, preferably in the form of granules. These granules may be uncoated or coated and may be prepared by any method known in the pharmaceutical art (e.g., wet or dry granulation methods). The drug granules so obtained can be combined with a combination of water-insoluble and water-soluble polymers.

Various equipment used for manufacturing the composition of the present invention, for instance, for blending, includes simple blending equipment known in the art can be used. For granulation, a mixer granulator or fluid bed processor or any other convenient granulating equipment can be used. For coating, conventional coating equipment can be used, but fluid bed coating is preferred. Aqueous and non-aqueous solvents can be used for the granulation and coating processes. For the production of bilayer tablets, a 27-station tablet machine (Cadmach-make) is used in which active and inactive formulations are compressed simultaneously to form a distinct bilayered tablet.

In another embodiment, the process for manufacturing a chewable bilayer tablet of cetirizine or its pharmaceutical acceptable salts comprises the steps of:

(a) preparing the active formulation, which further comprises the steps of
(i) sifting cetirizine, filler, coloring agent and disintegrant through suitable size sieves, and blending them together,
(ii) dissolving the binder in a suitable blend of aqueous and non aqueous solvents,
(iii) granulating the blend of step (a) (i) with the solution of step (a) (ii) in suitable equipment, and drying the granules so formed,
(iv) dissolving water-insoluble polymer and water-soluble polymer in suitable solvents separately and then mixing both the solutions,
(v) combining the granules of step (a) (iii) with the solution of step (a) (iv) in suitable equipment,
(vi) sifting sweetener, glidant, flavoring agents, coloring agents, fillers or bulking agents and disintegrants through suitable size sieves and blending them together,
(vii) passing lubricant through a suitable size sieve and blending with the contents of step (vi),
(b) preparing the inactive formulation
(i) sifting fillers, sweeteners, glidants, flavors, edible organic acids, coloring agents through suitable size sieves, and blending them together, optionally followed by suitable processing techniques to obtain granules,
(ii) sifting lubricants through a suitable sieve and mixing them with material of step (b) (i),
(c) forming a bilayer tablet of the active formulation and the inactive formulation
i) compressing the contents of step (a) (vii) and (b) (ii) simultaneously to form a chewable bilayer tablet of the active formulation and the inactive formulation as distinct layers.

If granulated mannitol is used in the above process as filler, then it can be prepared by granulating the mannitol with aqueous or non-aqueous solvent.

In addition to the above additives or excipients, the use of any conventional materials and procedures for preparing suitable dosage forms using the compositions of this invention known by those skilled in the art is envisioned.

Other features and embodiments of the invention will become apparent from the following examples which are given for illustration of the invention rather than for limiting its intended scope.

### Example 1

### Cetirizine Chewable Bilayer Tablet

### 1. Active Formulation

### 1.1 Certirizine Granules

| | | % w/w of Active |
|---|---|---|
| Ser. No. | Ingredients | Formulation |
| 1. | Cetirizine dihydrochloride | 3.33 |
| 2. | Sodium carbonate | 0.67 |
| 3. | Fumaric acid | 10 |
| 4. | Microcrystalline cellulose (Avicel PH101) | 13.33 |
| 5. | Aspartame | 6.67 |
| 6. | Polyvinylpyrrolidone (Kollidon-30) | 5.0 |
| 7. | Ethyl cellulose (Ethocel 4cps) | 13.33 |
| 8. | Hydroxypropyl methylcellulose | 5.0 |
| | (Pharmacoat-606) | |
| 9. | Polyethylene glycol 400 (Lutrol-400) | 3.33 |
| 10. | Purified water* | q.s |
| 11. | Isopropyl alcohol* | q.s |

*Not present in final formulation

### Manufacturing Procedure:

a) Cetirizine hydrochloride, polyvinylpyrrolidone (Kollidon-30) and sodium carbonate were dissolved in suitable quantity of purified water.
b) Fumaric acid, microcrystalline cellulose and aspartame were passed through suitable size mesh and were blended together.
c) The blend obtained in stage 1.1(b) was granulated with solution obtained in stage 1.1(a) in suitable equipment.
d) The blend obtained in stage 1.1 (c) was further granulated with isopropyl alcohol-water solution containing ethyl cellulose, hydroxypropyl methylcellulose and polyethylene glycol 400 in suitable equipment.
e) Granules were dried and passed through suitable size mesh.

### 1.2 Mannitol Granules

| | | % w/w of active |
|---|---|---|
| Ser. No. | Ingredients | formulation |
| 12. | Mannitol-25 | 16.67 |
| 13. | Ethyl cellulose- 4cps (Ethocel) | 3.33 |
| 14. | Isopropyl alcohol* | q.s. |

*Not present in final formulation

### Manufacturing Procedure:

f) Ethyl cellulose was dissolved in the required quantity of isopropyl alcohol.
g) Mannitol was passed through suitable size mesh and granulated with solution obtained in stage 1.2(f)

### 1.3 Extra-Granular Formulation

| | | % w/w of active |
|---|---|---|
| Ser. No. | Ingredients | formulation |
| 15. | Cetirizine granules of step 1.1(e) | 60.66 |
| 16. | Mannitol granules of step 1.2(g) | 20.0 |
| 17. | Magnesium stearate | 0.67 |
| 18. | Colloidal silicon dioxide (Aerosil-200) | 0.67 |
| 19. | Crospovidone (Polyplasdone XL) | 17.0 |
| 20. | Orange flavor | 0.17 |
| 21. | Vanilla cream flavor | 0.5 |
| 22. | FD&C Red no#40 | 0.33 |

### Manufacturing Procedure

h) Magnesium stearate, crospovidone (Polyplasdone XL), colloidal silicon dioxide, orange flavor, vanilla cream flavor, FD&C Red #40 were passed through suitable size mesh, and blended with mannitol granules of stage 1.2 (g).
i) The above-prepared blend was further blended with granules of stage 1.1(e).

### 2. Inactive Formulation

### 2.1 Intragranular (Granules)

| | | % w/w of Inactive |
|---|---|---|
| Ser. No. | Ingredients | Formulation |
| 1. | Beta-cyclodextrin (Kleptose ®) | 42.5 |
| 2. | Acesulfame potassium | 2.5 |
| 3. | Aspartame | 7.5 |
| 4. | Mannitol | 42.75 |
| 5. | Polyvinylpyrrolidone | 2.5 |
| 6. | Purified water* | q.s |

*Not present in final formulations

### Manufacturing Procedure:

a) Beta-cyclodextrin, acesulfame potassium, aspartame, and mannitol were passed through suitable size mesh and were blended together to get a uniform blend.
b) The blend obtained in stage 2.1(a) was granulated with an aqueous solution of polyvinylpyrrolidone.

### 2.2 Extragranular Formulation

| | | % w/w of Inactive |
|---|---|---|
| Ser. No. | Ingredients | Formulation |
| 7. | Granules of stage 2.1(b) | 97.75 |
| 8. | Magnesium stearate | 0.5 |
| 9. | Colloidal silicon dioxide (Aerosil-200) | 0.25 |
| 10. | Orange flavor | 0.25 |
| 11. | Vanilla cream flavor | 0.25 |
| 12. | FD&C Red no# 6 | 1.0 |

### Manufacturing Procedure:

c) Magnesium stearate, colloidal silicon dioxide, orange flavor and vanilla cream flavor were passed through suitable size mesh and were blended together to get a uniform blend.
d) The above prepared blend was mixed with granules of stage 2.1 (b).

### 3. Bilayer Tablet Compression

(a) The active blend of stage 1.3 (i) and the inactive blend of stage 2.2 (d) were compressed simultaneously as distinct layers to form a bilayer tablet using bilayer tableting machine.

### Example 2

### Cetirizine Chewable Bilayer Tablet

### 1. Active Formulation

### 1.1 Cetirizine Granules

| | | % w/w of Active |
|---|---|---|
| S. No | Ingredients | formulation layer |
| 1. | Cetirizine dihydrochloride | 4.16 |
| 2. | Microcrystalline cellulose (Avicel PH 101) | 25 |
| 3. | FD & C yellow # 6 | 0.20 |
| 4. | Croscarmellose sodium | 4.16 |
| 5. | Hydroxypropyl methylcellulose - 6cps | 6.87 |
| 7. | Ethylcellulose (Ethocel 4 cps) | 13.12 |
| 8. | Isopropyl alcohol* | q.s |
| 9. | Purified water* | q.s |

*Not present in final formulations

### Manufacturing Procedure:

a) Cetirizine hydrochloride, microcrystalline cellulose, croscarmellose sodium and FD & C yellow were passed through suitable size mesh.
b) All the above mentioned were mixed geometrically and blended together.
c) Hydroxypropyl methylcellulose was dissolved in a suitable quantity of purified water to produce granulating solution.
d) The blend of stage 1.1 (b) was granulated with solution obtained in stage 1.1 (c) in suitable equipment.
e) Hydroxypropyl methylcellulose and ethyl cellulose were dissolved in a suitable quantity of purified water and isopropyl alcohol respectively, then finally both solutions were mixed together to produce granulating solution.
f) The granules of stage 1.1 (d) were further granulated with solution obtained in stage 1.1 (e) in suitable equipment.
g) Granules were dried and passed through suitable size mesh.

### 1.2 Preparation of Mannitol Granules

| | | % w/w of Active |
|---|---|---|
| S. No | Ingredients | Formulation |
| 10. | Mannitol-25 | 20.83 |
| 11. | Croscarmellose sodium | 8.33 |
| 12. | FD & C yellow # 6 | 0.20 |
| 13. | Talc | 0.20 |
| 14. | Ethyl cellulose-4 cps (Ethocel) | 4.16 |
| 15. | Isopropyl alcohol* | q.s |

*Not present in final formulations

### Manufacturing Procedure:

h) Ethyl cellulose was dissolved in required quantity of isopropyl alcohol.
i) Talc was dispersed in the solution obtained in stage 1.2 (h)
j) Mannitol and croscarmellose sodium were passed through suitable size mesh and granulated with solution obtained in stage 1.2 (i).

### 1.3 Extra-Granular Active Formulation

| | | % w/w of Active |
|---|---|---|
| S. No | Ingredients | Formulation |
| 16. | Cetirizine granules of stage 1.1 (e) | 53.54 |
| 17. | Mannitol granules of stage 1.2 (h) | 34.16 |
| 18. | Acesulfame potassium | 1.66 |
| 19. | Colloidal silicon dioxide | 0.62 |
| 20. | Orange flavor | 0.42 |
| 21. | Croscarmellose sodium | 8.33 |
| 22. | FD & C yellow # 6 | 0.41 |
| 23. | Magnesium stearate | 0.83 |

### Manufacturing Procedure:

k) Acesulfame potassium, colloidal silicon dioxide, croscarmellose sodium, orange flavor and FD & C yellow # 6 were passed through suitable size mesh, and blended with granules of stage 1.1 (g) and mannitol granules of stage 1.2 (j).
l) The above-prepared blend was further blended with magnesium stearate.

Note: Molar ratio of mannitol to drug in the active formulation layer is 12.67.

### 2. Inactive Formulation

| | | % w/w of Inactive |
|---|---|---|
| S. No | Ingredients | Formulation |
| 1. | Beta-cyclodextrin | 40.48 |
| 2. | Spray dried mannitol (Pearlitol SD 200) | 47.62 |
| 3. | Acesulfame potassium | 1.90 |
| 4. | Colloidal silicon dioxide | 0.95 |
| 5. | Orange flavor | 0.24 |
| 6. | Aspartame | 2.86 |
| 7. | Fumaric acid | 4.76 |
| 8. | FD & C yellow # 6 | 0.24 |
| 9. | Magnesium stearate | 0.95 |

### Manufacturing Procedure:

a) Beta-cyclodextrin, spray dried mannitol, acesulfame potassium, colloidal silicon dioxide, orange flavor, aspartame, fumaric acid and FD & C yellow # 6 were passed through size mesh and were blended together to get a uniform blend.
b) The above-prepared blend was further blended with magnesium stearate.

### 3. Bilayer Tablet Compression

(a) The active blend of stage 1.3 (1) and the inactive blend of stage 2.1 (b) were compressed as separate layers to form a bilayer tablet using a bilayer tablet machine.

### Example 3

In order to assess the release of drug substance (cetirizine) from the drug product or dosage form, the bilayer tablet formulation of example 2 was subjected to a dissolution study. The dissolution profile from the bilayer tablet formulation of example 2 was compared with the dissolution profile from the commercially available cetirizine chewable tablet (Zyrtec® 10 mg) from Pfizer Labs, USA. The results from the study were presented in table 1 below. Dissolution parameters were as follows.

Dissolution apparatus: USP type II, RPM: 50
Dissolution medium: Water
Dissolution volume: 900 ml.
Temperature of dissolution medium: 37° C.±2° C.

TABLE 1

Percentage of drug (cetirizine) released from:

| Time (Min.) | Zyrtec ® tablet | Bilayer tablet of Example 2. |
|---|---|---|
| 10 | 86 | 94 |
| 20 | 98 | 97 |
| 30 | 100 | 97 |
| 45 | 100 | 97 |

From the above tabular data, it is clearly evident that the bilayer tablet formulation of invention (Ex. 2) has substantially the same dissolution profile as the Zyrtec® tablet.

### Example 4

In order to assess the stability of drug substance (cetirizine hydrochloride) in the drug product or dosage form, the bilayer chewable tablet formulation of example 2 was subjected to accelerated stability testing at 40° C.±2° C./75% RH±5% RH and observations were made during 3 months in aluminium/aluminium blisters for the percentage of unreacted cetirizine and degraded reaction products. The results were shown in table 2 below.

TABLE 2

| Study period | Cetiriizne (%) | Degraded reaction products (%) |
|---|---|---|
| Initial | 98.7 | 0.09 |
| After 1 month | 101.6 | 0.18 |
| After 2 months | 99.7 | 0.12 |
| After 3 months | 96.6 | 0.08 |

From the above tabular data it is clearly evident that the bilayer tablet formulation of the invention (Example 2) has a negligible amount of % w/w of undesired reaction products.

### Example 5

This example describes an in-vivo study which measured plasma cetirizine concentrations achieved after oral administration of reference Zyrtec® tablet and the test cetirizine chewable tablet formulation of example 2.

A comparative, randomized open label, two period, two treatment, two sequence, single dose, bioequivalence study of cetirizine hydrochloride 10 mg chewable tablets (Jubilant Organosys Ltd. India) and Zyrtec® 10 mg chewable tablets was carried out on 12 healthy adult human male subjects under fasting conditions. Plasma concentration of cetirizine was determined over a 24-hour period, after a single oral administration of the respective formulations.

This example demonstrates the ability of the formulation of example 2 (labeled amount 10 mg cetirizine) to provide bioavailability of cetirizine which is comparable to the bioavailability provided by Zyrtec® (labeled amount 10 mg cetirizine) as determined by the area under the curve (AUC) and Cₘₐₓ.

Individual plasma levels were measured at predetermined times utilizing a validated assay method employing LC-MS/MS instrumentation. Table 2 shows the pharmacokinetic parameters were estimated using winNonlin® software. The untransformed and natural log transformed pharmacokinetic parameters that were analyzed for statistical differences between test (example 2) and Zyrtec® using ANOVA, using the statistical software programme known as SAS Log transformed pharmacokinetic parameters, for both reference and test formulations.

TABLE 3
Pharmacokinetic Data

| | Reference | Test | | 90% confidence |
|---|---|---|---|---|
| | Product | Formulation | | interval (test vs |
| Parameter | (Zyrtec ®)-R | (Example 2) | T/R ratio | reference) |
| AUC₀₋₂₄ ng · h ·/ml | 2658.16 | 2670.16 | 100.44 | 97.17-103.83 |
| AUC_{0-∞} ng · h ·/ml | 2710.33 | 2720.62 | 100.38 | 97.18-103.67 |
| Cₘₐₓ ng/ml | 338.14 | 316.44 | 93.58 | 86.24-101.54 |
| Tₘₐₓ | — | — | — | — |

In view of the above, it is clearly evident that the formulation of the invention importantly provides bioavailability of cetirizine which is comparable or bioequivalent to Zyrtec®.

While several particular forms of the invention have been described, it will be apparent that various modifications and combinations of the invention detailed in the text can be made without departing from the spirit and scope of the invention.

1-17. (canceled)

18. An oral chewable bilayer tablet formulation comprising:
(a) an active formulation layer comprising cetirizine or a pharmaceutically acceptable salt thereof, and a combination of water-insoluble and water-soluble polymer and low molecular weight polyol, wherein said combination of water-soluble polymer and water-insoluble polymer is in a ratio of about 1:0.5 to about 1:5, and wherein the molar ratio of the said low molecular weight polyol to said cetirizine is more than 10, and
(b) an inactive formulation layer comprising beta-cyclodextrin and edible organic acids, wherein said beta-cyclodextrin is necessarily not in intimate contact with cetirizine or its pharmaceutically acceptable salt.

19. The formulation according to claim 18, wherein the water-insoluble polymer is selected from the group consisting of ethyl cellulose, polyvinyl acetate, neutral copolymer based on ethylacrylate and methylmetacrylate, copolymer of acrylic acid and methacrylic acid esters with quaternary ammonium compounds, polyvinyl acetate phthalate, hydroxypropyl methylcellulose phthalate, hydroxypropyl methylcellulose succinate, shellac, and a combination thereof.

20. The formulation according to claim 19, wherein the water-insoluble polymer is ethyl cellulose.

21. The formulation according to claim 18, wherein the water-soluble polymer is selected from the group consisting of hydroxypropyl methylcellulose, hydroxypropyl cellulose, hydroxyethyl cellulose, polyvinyl alcohol, polyvinylpyrrolidone, methylcellulose, carboxymethyl cellulose, polyethylene glycols, and a combination thereof.

22. The formulation according to claim 21, wherein the water-soluble polymer is hydroxypropyl methylcellulose.

23. The formulation according to claim 18, wherein the ratio of water-soluble polymer to water-insoluble polymer is about 1:1.5 to about 1:3.

24. The formulation according to claim 18, wherein the edible organic acid is selected from the group consisting of fumaric acid, citric acid, malic acid, succinic acid, ascorbic acid, maleic acid, tartaric acid, and a combination thereof.

25. The formulation according to claim 24, wherein the edible organic acid is fumaric acid.

26. The formulation according to claim 18, wherein the low molecular weight polyol has a molecular weight of less than 950 and is selected from the group consisting of mannitol, xylitol, sorbitol, dextrose, sucrose, lactose, and a combination thereof.

27. The formulation according to claim 26, wherein the low molecular weight polyol is mannitol.

28. The formulation according to claim 18, wherein the active formulation layer further comprises neutralizing agents, fillers, binders, sweetening agents, glidants, plasticizers, lubricants, disintegrating agents, flavoring agents or coloring agents.

29. The formulation according to claim 18, wherein the inactive formulation layer further comprises fillers, bulking agents, binders, sweetening agents, flavoring agents, glidants, disintegrants or lubricants.

30. The formulation according to claim 28, wherein the filler is selected from the group comprising microcrystalline cellulose, dicalcium phosphate, tricalcium phosphate, beta-cyclodextrin, lactose, mannitol, xylitol, sorbitol, dextrose or sucrose or combination thereof.

31. The formulation according to claim 29, wherein the filler is selected from the group comprising microcrystalline cellulose, dicalcium phosphate, tricalcium phosphate, beta-cyclodextrin, lactose, mannitol, xylitol, sorbitol, dextrose or sucrose or combination thereof.

32. The formulation according to claim 30, wherein the filler is mannitol.

33. The formulation of claim 31, wherein the filler is mannitol.

34. The formulation according to claim 18, wherein the beta cyclodextrin is directly compressible beta cyclodextrin.

35. A process for preparing the oral bilayer chewable tablet formulation according to claim 18, wherein the process comprises the steps of:
(a) preparing the active formulation layer comprising cetirizine, filler granules and other pharmaceutically acceptable excipients;
(b) preparing the inactive formulation layer comprising beta-cyclodextrin; and
(c) combining the active formulation layer and inactive formulation layer using a bilayer tablet machine to get the bilayered tablet.

36. The process according to claim 35, wherein the process comprises the steps of:
(a) preparing the active formulation layer comprising:
(i) granulating cetirizine, and other pharmaceutical excipients with an aqueous/non aqueous solution of binder and drying the granules so formed;
(ii) dissolving water-insoluble polymer and water-soluble polymer in a suitable solvent system;
(iii) combining the granules of step (i) with the solution of step (ii) in suitable equipment; and
(iv) mixing the contents of step (iii) with lubricant and other pharmaceutical excipients,
(b) preparing the inactive formulation layer comprising:
dry mixing cyclodextrin with other pharmaceutical excipients in suitable equipment, and
(c) preparing the bilayer tablet comprising,
compressing the resultant mixture obtained in step (iv) of step (a) and the resultant granules of step (b) to form a chewable bilayer tablet of said cetirizine.

37. The process according to claim 36, wherein step (b) further comprises forming granules from the resulting dry mixture.
